(19) United States
(12) Patent Application Publication
(10) Pub. No.: US 2015/0110719 A1
(43) Pub. Date: Apr. 23, 2015

(54) HYDROGEL BASED DELIVERY AND SENSOR

(71) Applicant: Health Line International Corp., Salt Lake City, UT (US)

(72) Inventors: Zhao Jie, Beijing (CN); Li Min She, Guangdong (CN)

(21) Appl. No.: 14/398,680

(22) PCT Filed: May 8, 2013
(86) PCT No.: PCT/US2013/040174

§ 371 Date: Nov. 3, 2014

Related U.S. Application Data
(60) Provisional application No. 61/644,237, filed on May 8, 2012.

(51) Int. Cl. A61B 5/1459; A61B 5/145; A61L 29/04; C07K 16/18; A61K 49/00

(52) U.S. Cl. CPC A61B 5/1459; C07K 16/18; A61K 49/0021; A61K 49/0054; A61L 29/048; A61B 5/14503; C07K 2317/76
USPC 424/9.6; 424/130.1; 600/309; 600/317; 600/310

(57) ABSTRACT

Described are catheters associated with a hydrogel for release of a molecule of interest. The molecule of interest may be an antibody. Further described are sensors useful for detecting the presence or amount of an analyte, and associated methods. A sensor for use in detecting the presence or amount of an analyte may comprise a catheter having one or more apertures. The sensor may also include means for detecting binding of the analyte to an antibody.

### PRIORITY CLAIM

This application claims the benefit of U.S. Provisional Application Ser. No. 61/644,237, filed May 8, 2012.

### TECHNICAL FIELD

The disclosure relates to the field of biotechnology and associated devices such as catheters. Specifically, disclosed are hydrogel-based systems for releasing a molecule of interest into a fluid space. Further disclosed are hydrogel-based sensors useful in, for example, detecting the level of analytes, and methods of using such sensors.

### BACKGROUND

A conventional approach for measuring analytes in the blood involves performing multiple in vitro assays to periodically screen for the analyte. However, this solution is imperfect because large spans of time occur when the body is not being monitored. It is possible that an important biological event may be missed such as a clinically relevant rate of change, or that treatment may be delayed by as long as the time interval between tests. Moreover, performing multiple assays is invasive to the patient, e.g., drawing blood every time a test is run.

A potential solution for detecting an analyte in the blood may be to insert a catheter with biosensing capabilities. However, such catheters take time to equilibrate, become saturated with analyte, and must be removed periodically. Thus, it is difficult or impossible to obtain continuous and instantaneous data over the period of time necessary for optimal care without multiple insertion and removal events, which may introduce more risk than if the patient was not monitored. For example, insertion and removal of a peripherally inserted central catheter (PICC) increases risk for air embolism, infection, phlebitis, catheter malposition, thrombus formation, nerve injury or irritation, leakage or catheter breakage.

These problems are of acute importance in, e.g., the monitoring of acute myocardial infarction (MI) patients. With conventional sensors, biochemical markers associated with MI (e.g., cardiac troponin) are detectable in the patient's blood stream about 3 to 8 hours from the onset of the condition. In the absence of other indications of the condition (e.g., electrocardiogram indicators, acute distress, etc.), a patient complaining of physical conditions associated with MI (e.g., chest pain) is typically observed for up to 12 hours to determine whether an infarction is the cause of the symptoms. Cardiac marker assays are typically performed serially at 4-8 hour intervals in order to detect a recent infarct. Due to the relatively long time periods between assays, a true infarction patient with biological signs of infarction may, as a result, wait for many hours before the signs are detected. Consequently, a delay exists in providing timely therapy to the patient.

Another complication of in vivo analyte detection is delivery of an appropriate biosensor. When the analyte is a protein, for example, the biosensor is usually antibody-based, since the interaction of an antibody with an antigen is very specific. Detection of other analytes may require release of other molecules of interest. Continuous in vivo release of a molecule of interest at a point of detection is challenging, however.

It would be desirable to provide a catheter-based system for continuous release of a molecule of interest. It would also be desirable to provide a sensor that continuously detects the presence of an analyte, such as troponin, in a fluid space (e.g., blood) to allow for instantaneous and continuous detection of analyte concentrations.

### DISCLOSURE

In embodiments, a catheter associated with a hydrogel for releasing a molecule of interest into a fluid space is disclosed, wherein the catheter is in fluid communication with the fluid space, the hydrogel is substantially contained within at least a portion of the catheter; and the molecule of interest is substantially dispersed within the hydrogel.

In certain embodiments, a molecule of interest is an antibody, an antibody fragment, or combinations thereof. In certain embodiments, a catheter for releasing a molecule of interest further comprises a means for detecting an analyte present in the fluid space.

In some embodiments, a method is provided for releasing a molecule of interest into a fluid space, the method comprising: positioning a catheter within a fluid space, wherein the catheter comprises a hydrogel substantially contained within at least a portion of the catheter, and a molecule of interest substantially dispersed within the hydrogel; and releasing the molecule of interest into the fluid space.

In embodiments, the fluid space may be interior to a subject. In some embodiments, the fluid space comprises blood. In some embodiments, the method further comprises detecting an analyte present in the fluid space.

In embodiments, a catheter is provided for detecting the presence or amount of an analyte, the catheter comprising: a compartment having one or more apertures, wherein the compartment is in fluid communication with a surface of the catheter and the one or more apertures; a hydrogel disposed within the compartment and in fluid communication with the one or more apertures; molecule(s) comprising antibody, antibody fragment, or combinations thereof specific for the analyte dispersed within the hydrogel; a source of light and/or radiation in operable contact with the hydrogel through at least one optic fiber; and a means for detecting binding of the analyte to the antibody, antibody fragment, or combination thereof.

### BRIEF DESCRIPTION OF THE FIGURES

FIG. 1 illustrates an embodiment of a cross-section of a catheter associated with a hydrogel having antibody or antibody fragments dispersed in the hydrogel, a single aperture and, optionally, an optic fiber.

FIG. 2 illustrates an embodiment of a cross section of a catheter associated with a hydrogel having antibody or antibody fragments dispersed in the hydrogel, multiple microneedle apertures and, optionally, an optic fiber.

FIG. 3 illustrates an embodiment of a sensor system showing components exterior to the catheter and including a source of light and/or radiation.

FIG. 4 illustrates an embodiment of a sensor system showing components wherein the catheter is exterior to the source of the sample.

FIG. 5 illustrates an embodiment of a catheter, having a hydrogel substantially contained within a catheter, a means for detecting an analyte and, optionally, an optic fiber within the catheter.

FIG. 6 illustrates an embodiment of an ex vivo catheter, having a hydrogel substantially contained within the catheter, and means for detecting and analyzing an analyte.

FIG. 7 is a graph illustrating the release trend for IgG from a hydrogel.

### MODE(S) FOR CARRYING OUT THE INVENTION

In one aspect, disclosed herein are catheters useful for releasing a molecule of interest into a fluid space. Further disclosed are methods for releasing a molecule of interest into a fluid space. In embodiments, a catheter for releasing a molecule of interest into a fluid space comprises: a catheter in fluid communication with the fluid space, wherein the catheter comprises one or more apertures; a hydrogel, wherein the hydrogel is substantially contained within a portion of the catheter; and a molecule of interest, wherein the molecule of interest is substantially dispersed within the hydrogel.

In another aspect, disclosed herein are sensors for detecting the presence or amount of an analyte. Further disclosed are methods for detecting the presence or amount of an analyte.

“Catheters,” as used herein, means and includes various flexible and inflexible tubes that are inserted into a body cavity, duct or vessel to treat or monitor medical conditions, administer drugs, gases, or fluids to a subject, or allow drainage and sampling of body fluids. Thus, catheters include, but are not limited to, peripherally inserted central catheters (PICCs), central catheters, venous catheters, dialysis catheters, indwelling catheters (e.g., Foley catheter), lubricating catheters, and umbilical lines. Catheters may be positioned into a third space, for example, into a blood vessel, to sample blood and measure the levels of specific analytes. Catheters may remain in the body for extended periods of time, allowing for prolonged, continuous monitoring, sampling, and/or administration of desired agents.

Various modifications and accessories may be associated with catheters. For example, guide wires, optic fibers, stents, CCDs, light sources and radiation sources may be passed through a catheter to a site of interest inside the fluid space. One or more surfaces of a catheter may be coated (e.g., coated with lubricant, anti-microbial agents, or antithrombotic agents).

As used herein, a “hydrogel” is a network of water-soluble, hydrophilic polymer chains, sometimes found as a colloidal gel, in which water is the dispersion medium. Examples of polymers that may be used to form hydrogels include, but are not limited to, polyvinyl alcohols, acrylic acids, poly acrylates, pHEMA, pMMA, DMAEME, PEG, collagen, polyethylene oxide, polyAMPS, polyvinylpyrrolidone, poly-carboxylic acids, cellulosic polymers, gelatin, maleic anhydride polymers, polyamides, and combinations thereof.

A hydrogel for use in some embodiments may comprise between 20% and 95% water, or 20%, 25%, 30%, 35%, 40%, 45%, 50%, 55%, 60%, 65%, 70%, 75%, 80%, 85%, 90%, or 95% water. In certain embodiments, the hydrogel may be inserted into a subject as a xerogel or lacking the full amount of water it may later adsorb. In such cases, the hydrogel may draw water from the solution in which it is placed in order to reach its full water holding capacity.

Hydrogels may be useful for the encapsulation and delivery of various compounds. In particular, hydrogels may be used for absorption and delivery of certain water-soluble and alcohol-soluble active agents. In embodiments, the matrix of a hydrogel contains various molecules of interest, including pharmaceutical agents, proteins (e.g., antibodies, enzymes), vitamins, oils, or other compounds. In embodiments, the molecules of interest may be substantially dispersed in the hydrogel. In certain embodiments, a molecule of interest may be an antibody. In some embodiments, an antibody present in the matrix of a hydrogel may be released from the hydrogel by, for example, diffusion into the surrounding fluid space. In some embodiments, the antibody is capable of binding an analyte present in the fluid space. In other embodiments, an antibody capable of binding an analyte substantially dispersed in the hydrogel.

In embodiments, a catheter for releasing a molecule of interest into a fluid space is associated with a hydrogel. In some embodiments, the hydrogel may be substantially contained within a portion of the catheter. In some embodiments, a hydrogel is substantially contained in a fixed space on one or more surfaces of the catheter (e.g., a compartment, bounded region, or otherwise enclosed or partially enclosed region within, on, or inside the catheter). In certain embodiments, the fixed space may contain one or more apertures to allow diffusion of materials between the hydrogel and the fluid space.

In some embodiments, a hydrogel coats one or more surfaces of the catheter. In certain embodiments, the hydrogel coating is disposed on the inner surface of a catheter. In certain embodiments, a hydrogel coating is disposed at or near the distal end of the catheter. For example, the hydrogel coating may be disposed as a coating on the inner surface near the terminus of an indwelling catheter within the body, to allow release of a molecule of interest and/or detection of an analyte at a target region of interest.

In some embodiments, a hydrogel is substantially contained in a sensor. In certain embodiments, the sensor is positioned at or near the distal end of the catheter. In certain embodiments, a sensor may contain one or more apertures to allow diffusion of materials between the hydrogel and the fluid space.

An “analyte,” as used herein, is any substance or chemical that is of interest in an analytical procedure. More specifically, an analyte may refer to a substance that is present in the body, is capable of being measured or detected, and has clinical significance. In some embodiments, an analyte is any molecule to which an antibody may be generated. Examples of analytes include, but are not limited to, troponin, atrial natriuretic peptide, brain natriuretic peptide, C-reactive protein, fibrinogen, D-dimer, lipoprotein-associated Lp-PLA₂, homocysteine, adiponectin, soluble CD40 ligand, cholesterol, myeloperoxidase, placental growth factor, and ischemia modified albumin.

In one aspect, disclosed herein are methods and catheters for use in in vivo applications. For example, methods disclosed herein for detecting the presence or amount of an analyte may comprise placement of a catheter inside an interior fluid space of a subject (e.g., a blood vessel). In another aspect, disclosed herein are methods and catheters for use in ex vivo applications. In some embodiments, a catheter associated with a hydrogel for releasing a molecule of interest into a fluid space is positioned outside the body. For example, a hydrogel may be substantially contained on an inner surface of a dialysis catheter external to the body. In certain embodiments, an ex vivo catheter further comprises means for detecting an analyte present in the fluid space.

Further disclosed herein are sensors for detecting the presence or amount of an analyte in a fluid space. In certain embodiments, the sensor comprises a hydrogel. The fluid space may be one that is present inside a living subject during the detection. Examples of fluids within a fluid space include, but are not limited to, blood, lymph, interstitial fluid, urine, gastrointestinal juices, and cerebrospinal fluid (CSF).

Biomedical sensors can be used to report the presence and/or concentration of a wide variety of analytes. A sensor, sensor head, and/or probe according to embodiments herein is inserted into, e.g., a blood vessel, of a subject. When the analyte is a protein, the sensor is usually antibody-based, since the interaction of an antibody with the antigen is very specific. Typically, at least the complementary determining region of one or more antibodies is dispersed in the hydrogel. The complementary determining region is selected for its ability to specifically bind to an analyte. In certain embodiments, the complementary determining region may be attached, linked, or conjugated to other molecules or surfaces. Examples of other molecules and surfaces include, but are not limited to, other antibody regions, linkers, spacers, substrates, detectable markers, labels, enzymes, tags, and combinations thereof. Examples of detectable markers include, but are not limited to, fluorophores.

In another aspect, methods are disclosed for detecting the presence of an analyte present in a fluid space. In certain embodiments, the binding of the analyte to an antibody or antibody fragments may be detected.

In some embodiments, a method for detecting the presence or amount of an analyte comprises:
inserting a catheter associated with a hydrogel into a fluid space of a subject, wherein the hydrogel is substantially contained within at least a portion of the catheter;
binding analyte present in the fluid to a molecule of interest dispersed within the hydrogel, at an interface of the hydrogel and the fluid, or in the fluid proximal to the hydrogel,
wherein the molecule of interest is selected from the group consisting of an antibody, an antibody fragment, or combinations thereof;
detecting the binding of the analyte to the antibody, antibody fragment, or combination thereof;
allowing diffusion of antibody, antibody fragment, or combination thereof into the fluid so as to release the bound antibody, antibody fragment, or combination thereof and to allow the diffusion of unbound antibody, antibody fragment, or combination thereof to the interface of the hydrogel and the fluid;
binding analyte present in the fluid to the unbound antibody, antibody fragment, or combination thereof; and
detecting the binding of the analyte to the antibody, antibody fragment, or combination thereof.

In some embodiments, an antibody or antibody fragment contained in a hydrogel is exposed to the fluid in a fluid space through one or more apertures in the catheter, sensor, sensor head, and/or probe and binds analyte present in the blood. In some embodiments, detection utilizes a change in fluorescence to detect the binding of the analyte to the antibody or antibody fragments. The bound complementary determining regions of the antibody or antibody fragments may then be allowed to diffuse out of the hydrogel and into the fluid, thus removing the bound complementary determining regions. Further complementary determining regions in the hydrogel may then diffuse to the interface between the hydrogel and the fluid, thus presenting additional opportunities for complementary determining regions to bind the analyte. The freshly bound complementary determining regions may then be detected. In certain embodiments, the detection may be performed continuously or at one or more time points. In certain embodiments, a release profile of the complementary determining regions may be used with the detection of bound complementary determining regions to provide a concentration of the analyte in the fluid.

In certain embodiments, the complementary determining region may be released into the fluid from one or more apertures in the sensor by diffusion. The release rate of the complementary determining regions into the fluid will be a function of the type of hydrogel (e.g. polymer(s) used and water content), the concentration of the complementary determining regions therein, and the flow of fluid past the one or more apertures. In certain embodiments, a release profile may be ascertained prior to insertion of the device, providing information regarding the concentration of complementary determining regions at the interface between the hydrogel and the fluid over time.

Disclosed herein are means for detecting the interaction of an antibody or antibody fragment with an analyte within the hydrogel, at the interface between the hydrogel and the fluid, or in the fluid proximal to the interface between the hydrogel and the fluid. Means for detecting may include cameras, such as a CCD camera, optic fibers, optical waveguides, lenses, prisms, filters, photomultipliers, waveguides, beam splitters, processors, metal layers, sources of light or radiation, and combinations thereof such that the means may detect, without limitation, changes in the fluorescent emission of a fluorophore, fluorescence of the analyte, auto fluorescence, changes in the wavelengths or intensity of emitted or absorbed radiation and/or colorimetric change. The source of light and/or radiation may include, but is not limited to, lasers, LEDs, and lamps.

In some embodiments, one or more layers of a degradable substrate are deposited on the surface of or within a cavity on the surface of a probe or sensor head. In certain embodiments, the layers of the degradable substrate may be deposited on a surface within the lumen of a catheter. In further embodiments, the layers of the degradable substrate are deposited on the sensing region of a scaffold. The catheter, probe or sensor head may have one or more apertures which allow contact between a hydrogel disposed within the sensor head and a solution in which the catheter, probe or sensor head is placed. In certain embodiments, the one or more apertures may be microneedles.

In certain embodiments, layers of the hydrogel may be 10 nm, 20 nm, 30 nm, 49 nm, 50 nm, 60 nm, 70, nm, 80 nm, 90 nm, 100 nm, 110 nm, 120 nm, 130 nm, 140 nm, 150 nm, 160 nm, 170 nm, 180 nm, 190 nm, or 200 nm in thickness; less than 200 nm in thickness; or for 10-100 nm in thickness.

Particular embodiments are described with reference to the accompanying drawings. In the following description, well-known functions or constructions are not described in detail to avoid obscuring the present disclosure.

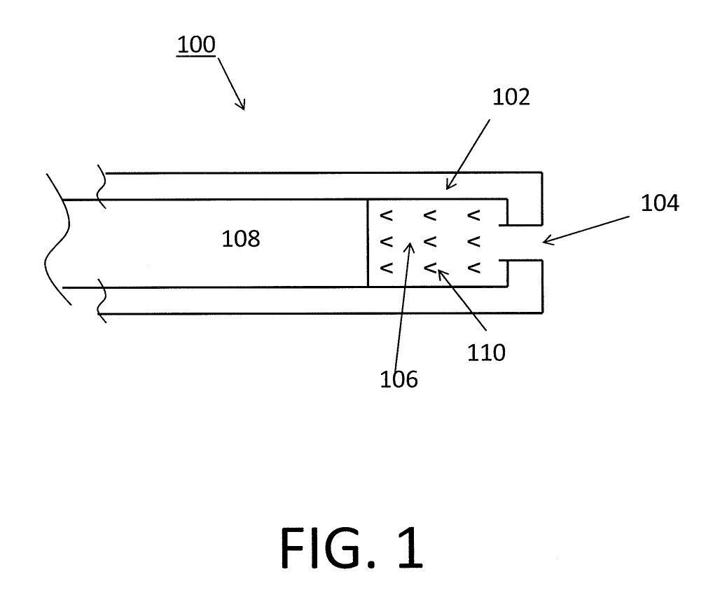

FIG. 1 depicts a cross-section of one embodiment of a catheter (100). The catheter (100) includes a catheter head (102) having an aperture (104) in fluid communication with a hydrogel (106) and, optionally, an optic fiber (108). Dispersed within the hydrogel (106) are antibody or antibody fragments (110).

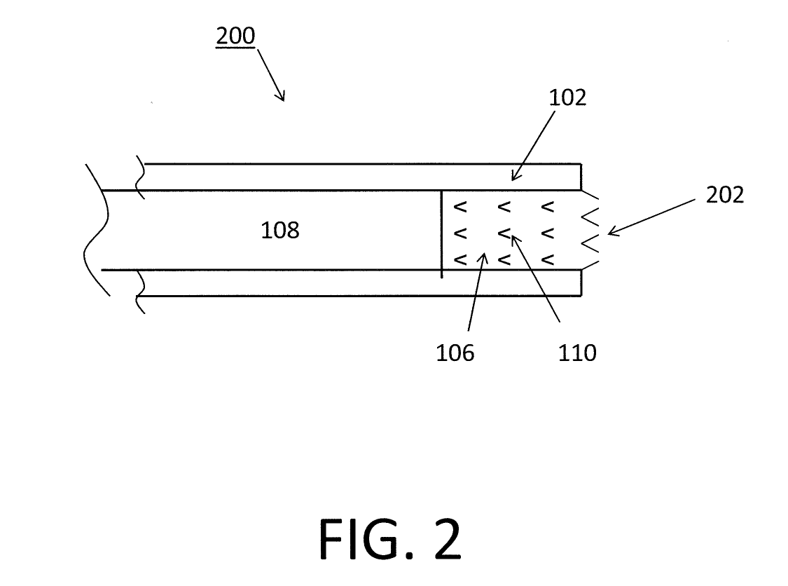

FIG. 2 depicts a cross-section of another embodiment of a catheter (200). The catheter (200) includes a catheter head (102) having multiple microneedle apertures (202) in fluid communication with a hydrogel (106) deposited within the catheter head (102) and, optionally, an optic fiber (108). Dispersed within the hydrogel (106) are antibodies or antibody fragments (110).

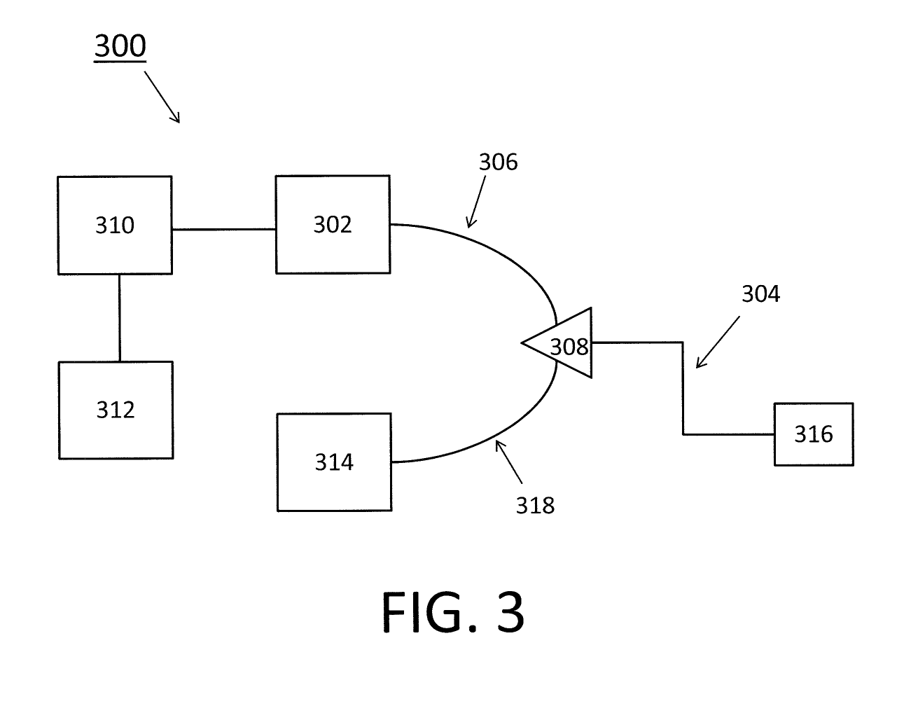

FIG. 3 depicts an embodiment of a sensor system (300). The sensor system (300) includes a catheter (316) connected to a camera (302) via a first optic fiber (304), second optic fiber (306), and beam splitter (308). A catheter (316) may be any embodiment of a catheter comprising a hydrogel and at least one aperture. A camera (302) is operably linked to a processing unit (310) and a display (312). The catheter (316) is also connected to a source of light and/or radiation (314) via a first optic fiber (304), beam splitter (308), and third optic fiber (318).

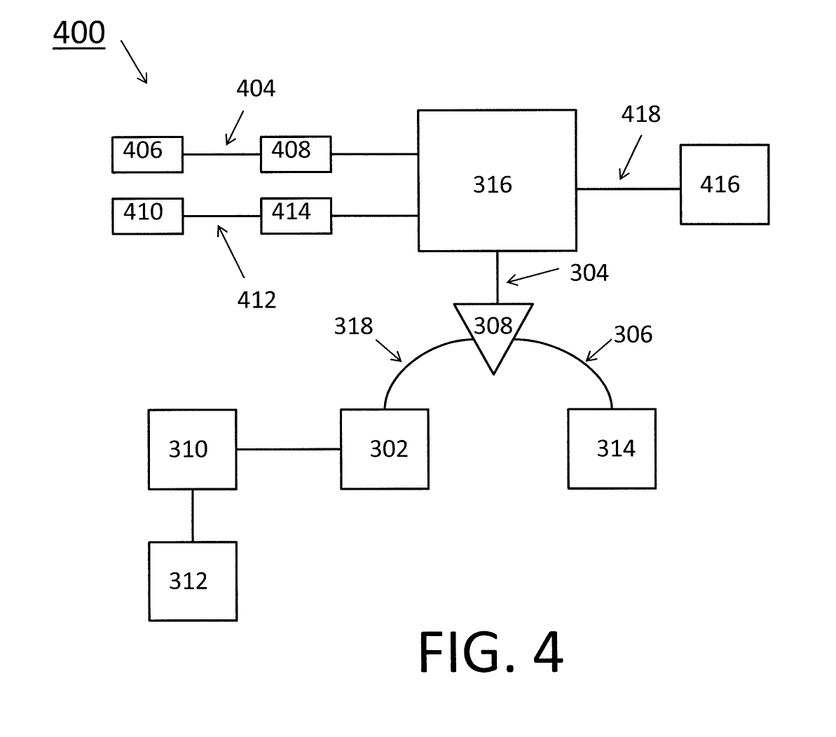

FIG. 4 depicts another embodiment of a sensor system (400). The sensor system (400) includes a catheter (316) connected to a beam splitter (308) via a first optic fiber (304). The catheter (316) may be any embodiment of a catheter comprising a hydrogel. A source of light or radiation (314) is connected to a beam splitter (308) through the second optic fiber (306). A camera (302) is connected to the beam splitter (308) through a third optic fiber (318). The camera (302) is operably linked to a processing unit (310) and a display (312). A PICC line (404) connects a source of fluid to be analyzed (406) to a catheter (316) via a first pump (408). In certain embodiments, the source of fluid to be analyzed (406) may be a reservoir or a living subject from which a PICC line (404) draws a sample to be analyzed, which is provided to the catheter (316) via the first pump (408). A wash reservoir (410) is connected to a catheter (316) via a first connecting tube (412) and a second pump (414). In certain embodiments, a sensor system (400) may also contain a reservoir of layer removal solution connected to the catheter via a connecting tube and a third pump (not shown). In embodiments, the catheter (316) may also be connected to a waste reservoir (416) via a second connecting tube (418).

In the operation of certain embodiments, a PICC line (404) may be used to draw a sample from the source of fluid to be analyzed (406) and provided to the catheter (316). The level of binding of an analyte to a molecule of interest released by the catheter (316) may be determined using a source of light and/or radiation (314), beam splitter (308), first optic fiber (304), second optic fiber (306), camera (302), third optic fiber (318), processing unit (310), and display (312). Optionally, before detection, unbound sample may be washed from the catheter (316) by providing a wash solution from a wash reservoir (410) to a catheter (316) via a first connecting tube (412) and a second pump (414). Sample and wash solution removed from the catheter (316) may be collected in a waste reservoir (416) via a second connecting tube (418). Optionally, after detection, the catheter may be washed, as described, to remove any unbound sample. After washing as described the catheter (316) is thus prepared to receive another sample. In certain embodiments, the catheter (316) may be left in contact with a wash solution for a period of time so as to elute and/or remove bound antigens and make available new, unbound, complementary determining regions before presenting the catheter (316) with a new sample.

In certain embodiments, a first pump (408) and second pump (414) may be a single pump that is connected to each of a first connecting tube (412) and a second connecting tube (418). In such embodiments, the sensor system (400) may include a valve structure or other means for selecting which of the connecting tubes to be drawn from for a given pumping action. In certain embodiments, the components may be controlled by a central processor which directs the function and/or action of optional valves, the pump(s), source of light and/or radiation (314), camera (302), processing unit (310), and display (312).

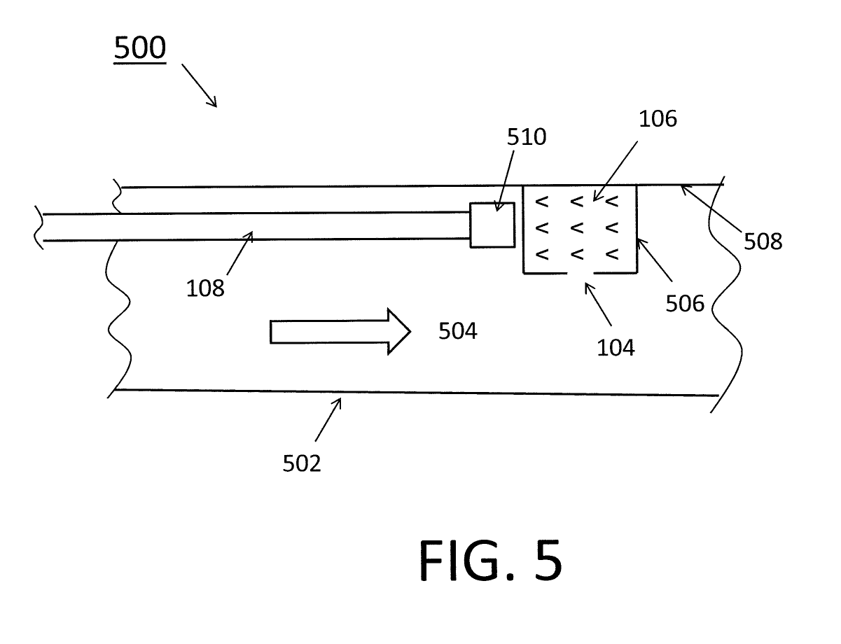

FIG. 5 depicts a catheter system (500) for use in certain embodiments. In the catheter system (500), a catheter (502) is shown, in fluid communication with a fluid space (504). The path of fluid flow is illustrated by a large arrow. A hydrogel (106) is contained within a compartment (506) in fluid communication with an inner surface (508) of the catheter (502) and an aperture (104). A molecule of interest (not illustrated) is dispersed within the hydrogel (106). An optic fiber (108) is connected to a means for detecting an analyte (510).

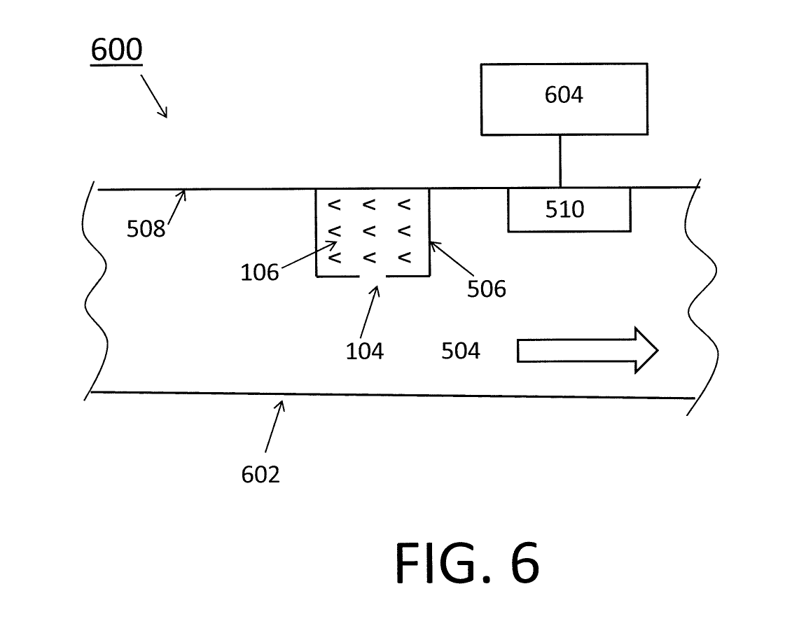

FIG. 6 depicts an ex vivo catheter system (600) for use in certain embodiments. In the ex vivo catheter system (600), an ex vivo catheter (602) is shown, in fluid communication with a fluid space (504). The path of fluid flow is illustrated by a large arrow. A hydrogel (106) is contained within a compartment (506) in fluid communication with the inner surface (508) of the catheter (502) and an aperture (104). A molecule of interest (not illustrated) is dispersed within the hydrogel (106). A means for detecting an analyte (510) is connected to an analyzer (604) external to the ex vivo catheter (602).

While aspects of this invention have been described in certain embodiments, they can be further modified within the spirit and scope of this disclosure. This application is therefore intended to cover any variations, uses, or adaptations of embodiments of the invention using its general principles. Further, this application is intended to cover such departures from the present disclosure as come within known or customary practice in the art to which these embodiments pertain and which fall within the limits of the appended claims.

The present invention is further described in the following examples, which are offered by way of illustration and are not intended to limit the invention in any manner.

### EXAMPLES

### Example 1

### Antibody Loading and Release

Antibody-loaded hydrogels were prepared by mixing 23.75 g of IgG with 25 g of dimethyl silicone oil for 30 minutes at 100 rpm. Polysiloxane was added, and the resulting mixture was blended for an additional 30 min at 50 rpm, yielding a silicon-shaped gel polysiloxane-antibody component (A) of approximately 50 g. The A component was then blended in a 1:1 ratio with a B component silicon-hydrogen sample (100 g), and 10 silicon setting gels (20 g/sample) were obtained. The pH of the setting gels was adjusted to 7.4. A standard concentration curve was obtained by dissolving lyophilized IgG powder in PBS at 10, 50, 100, and 150 mg IgG per liter of solution. A standard concentration curve formula was calculated.

Sample gel was soaked in PBS buffer at 37 C, and 1 mL samples obtained from the buffer were analyzed using high-performance liquid chromatography (HPLC) (Agilent 1200) at 280 nm, at a 0.4 mL/min flow rate. Timing of sample collection is illustrated in Table 1, below. An “x” indicates days when testing was conducted on a given sample.

TABLE 1
Test Samples and Timing.

| Sample | | | | | | |
|---|---|---|---|---|---|---|
| No. | Day 1 | Day 2 | Day 5 | Day 10 | Day 15 | Note |
| 1 | xx | xx | xx | | | |
| 2 | xx | xx | xx | | | |
| 3 | xx | xx | xx | | | Normal temp |
| | | | | | | preservation |
| 4 | xx | xx | xx | xx | | |
| 5 | | | xx | xx | | |
| 6 | | | xx | xx | | |
| 7 | | | xx | xx | | |
| 8 | | | xx | xx | xx | |
| 9 | | | xx | xx | xx | |
| 10 | xx | xx | xx | xx | xx | |

The concentration of IgG in the sample was calculated using the following formula:

X=CV×100/m×1000;

where X=IgG concentration, in grams IgG per 100 g sample;

C=content of fluid IgG (mg/mL);

V=sample volume set capacity (mL);

and m=sample mass (g).

Table 2 illustrates test results.

TABLE 2
IgG concentration in Released Sample.

| | Test | Day 1 | Day 2 | Day 5 | Day 10 | Day 15 | Theoretical | Difference |
|---|---|---|---|---|---|---|---|---|
| Sample | value (g) | (g) | (g) | (g) | (g) | (g) | total (g) | (%) |
| 1 | 2.182 | 0.727 | 0.581 | 0.274 | | | 2.375 | 8.12 |
| 2 | 2.253 | 0.684 | 0.552 | 0.203 | | | 2.375 | 5.1 |
| 3 | 2.217 | 0.932 | 0.723 | 0.027 | | | 2.375 | 6.6 |
| 4 | 2.171 | 0.679 | 0.562 | 0.298 | 0.091 | | 2.375 | 8.5 |
| 5 | 2.246 | | | 0.665 | 0.282 | | 2.375 | 5.4 |
| 6 | 2.27 | | | 0.747 | 0.224 | | 2.375 | 4.4 |
| 7 | 2.249 | | | 0.742 | 0.279 | | 2.375 | 5.3 |
| 8 | 2.183 | | | 0.692 | 0.284 | 0.071 | 2.375 | 9.9 |
| 9 | 2.201 | | | 0.682 | 0.248 | 0.062 | 2.375 | 7.3 |
| 10 | 2.225 | 0.663 | 0.541 | 0.214 | 0.082 | 0.047 | 2.375 | 6.3 |

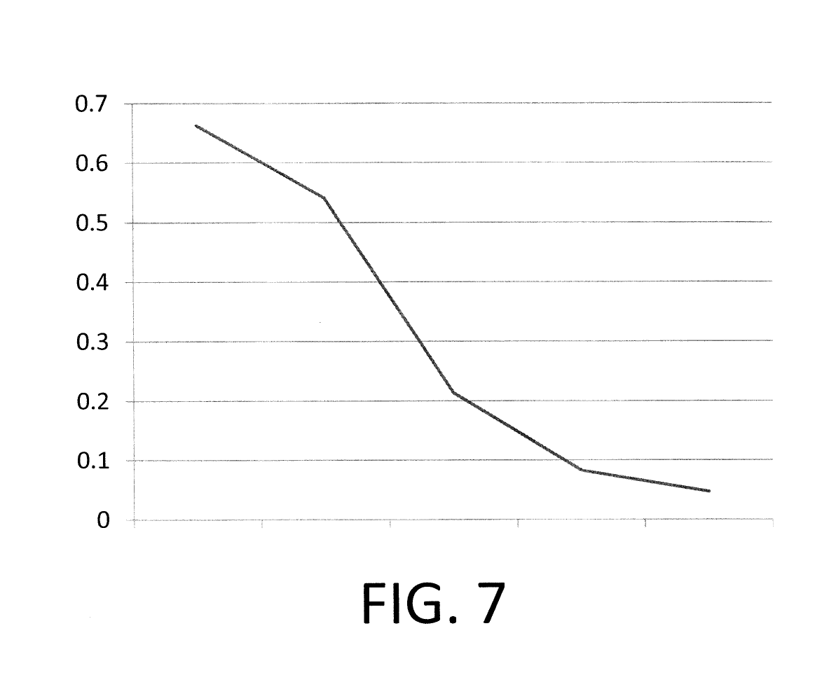

FIG. 7 illustrates the release trend for sample 10.

### Example 2

### Antibody Activity

Experiments were performed to determine if the antibodies released from the hydrogel of Example 1 maintained their activity, with respect to their antigen-specific binding capacity.

A peristaltic pump was used to pass buffer solution over IgG-loaded hydrogel samples prepared according to the method previously described. See Example 1. A flow rate of 0.2 L/hr for 30 minutes was used to simulate the process of loading and release. 5 mL samples were obtained from the buffer solution. ELISA analysis was performed using a BLAcore 1000 biosensor (Pharmacia BiosensorAB, Sweden), and automatic microplate reader (Sunrise Ruishi Di Ken), IgG antibody, and IgG-specific antigens as controls, following ISO17025. An automatic microplate reader method was used and OD values obtained, with OPD as a chromogenic substrate.

TABLE 3
Table 3 illustrates test results.
The activity of released IgG was determined.
ELISA Analysis of IgG Binding.

| Item | OD/Standard | Pass (yes/no) | Active? |
|---|---|---|---|
| 1 | 0.923/0.947 | yes | Active |
| 2 | 0.877/0.912 | yes | + Active |
| 3 | n/a | yes | + Active |
| 4 | n/a | yes | + Active |
| 5 | 1.128/1.082 | yes | + Active |
| 6 | 0.892/0.773 | yes | + Active |
| 7 | n/a | no | no |
| 8 | n/a | no | no |
| 9 | 0.982/0.874 | yes | + Active |
| 10 | 1.127/1.232 | Yes, low temp | + Active |
| 11 | 0.822/0.784 | yes | + Active |
| 12 | 0.767/0.655 | yes | + Active |
| 13 | n/a | yes | + Active |
| 14 | n/a | n/a | n/a |
| 15 | 0.912/0.887 | yes | + Active |

These results illustrate that IgG released from a hydrogel system maintains activity, with respect to its antigen-specific binding capacity (12 of 14 samples passed).

What is claimed is:

1. A catheter associated with a hydrogel for releasing a molecule of interest into a fluid space, wherein:
the catheter is in fluid communication with the fluid space;
the hydrogel is substantially contained within at least a portion of the catheter; and
the molecule of interest is substantially dispersed within the hydrogel.

2. The catheter of claim 1, wherein the molecule of interest is selected from the group consisting of an antibody, an antibody fragment, or combinations thereof.

3. The catheter of claim 1, wherein the fluid space comprises blood.

4. The catheter of claim 2, wherein the antibody, antibody fragment, or combinations thereof are specific for binding an analyte present in the fluid space.

5. The catheter of claim 2, wherein the antibody, antibody fragment, or combinations thereof are specific for binding an analyte substantially dispersed within the hydrogel.

6. The catheter of claim 1, further comprising a means for detecting an analyte present in the fluid space.

7. The catheter of claim 2, wherein the antibody, antibody fragment, or combinations thereof are conjugated with a label.

8. The catheter of claim 7, wherein the label is a fluorphore.

9. The catheter of claim 6, wherein the means for detecting the analyte is selected from the group consisting of a camera, CCD, optic fiber, lens, prism, filter, photomultiplier, waveguide, beam splitter, processor, metal layer, source of light or radiation, and combinations thereof.

10. The catheter of claim 6, wherein the means for detecting the analyte comprises measuring changes in the fluorescent emission of a fluorophore, measuring fluorescence of the analyte, measuring changes in the wavelengths or intensity of emitted or absorbed radiation from the analyte, or combinations thereof.

11. A method for releasing a molecule of interest into a fluid space, the method comprising:
positioning a catheter within a fluid space, wherein the catheter comprises:
a hydrogel substantially contained within at least a portion of the catheter,
and a molecule of interest substantially dispersed within the hydrogel; and
releasing the molecule of interest into the fluid space.

12. The method of claim 11, wherein the fluid space comprises blood.

13. The method of claim 11, wherein the molecule of interest is selected from the group consisting of an antibody, an antibody fragment, or combinations thereof.

14. The method of claim 13, wherein the antibody, antibody fragment, or combinations thereof are specific for binding an analyte present in the fluid space.

15. The method of claim 13, wherein the antibody, antibody fragment, or combinations thereof are specific for binding an analyte dispersed within the hydrogel.

16. The method of claim 13, wherein the antibody, antibody fragment, or combinations thereof are conjugated with a label.

17. The method of claim 16, wherein the label is a flurophore.

18. The method of claim 13, further comprising detecting an analyte present in the fluid space.

19. The method of claim 18, wherein detecting an analyte comprises measuring changes in the fluorescent emission of a fluorophore, measuring fluorescence of the analyte, measuring changes in the wavelengths or intensity of emitted or absorbed radiation from the analyte, or combinations thereof.

20. A method for detecting the presence or amount of an analyte, the method comprising:
inserting the catheter of claim 1 into a fluid space of a subject;
binding analyte present in the fluid to the molecule of interest at an interface of the hydrogel and the fluid, or in the fluid proximal to the hydrogel,
wherein the molecule of interest is selected from the group consisting of an antibody, an antibody fragment, or combinations thereof;
detecting the binding of the analyte to the antibody, antibody fragment, or combination thereof;
allowing diffusion of an antibody, antibody fragment, or combination thereof into the fluid so as to release the bound antibody, antibody fragment, or combination thereof and to allow the diffusion of unbound antibody, antibody fragment, or combination thereof to the interface of the hydrogel and the fluid;
binding analyte present in the fluid to the unbound antibody, antibody fragment, or combination thereof; and
detecting the binding of the analyte to the antibody, antibody fragment, or combination thereof.

21. The method according to claim 20, wherein detecting the binding of the analyte comprises detecting a change in the fluorescence of a fluorophore conjugated to the antibody, antibody fragment, or combination thereof.

22. A catheter for detecting the presence or amount of an analyte, the catheter comprising:
a compartment having one or more apertures, wherein the compartment is in fluid communication with a surface of the catheter and the one or more apertures;
a hydrogel disposed within the compartment and in fluid communication with the one or more apertures;
molecule(s) comprising antibody, antibody fragment, or combinations thereof specific for the analyte dispersed within the hydrogel;
a source of light and/or radiation in operable contact with the hydrogel through at least one optic fiber; and
a means for detecting binding of the analyte to the antibody, antibody fragment, or combination thereof.

23. The sensor of claim 22, wherein the means for detecting the binding of the analyte to the antibody, antibody fragment, or combination thereof comprises an optic fiber and a camera or CCD.

24. The sensor of claim 22, wherein the antibody, antibody fragment, or combination thereof is conjugated to a label.

25. The sensor of claim 24, wherein the label is a fluorophore.
